(19) United States
(12) Patent Application Publication
(10) Pub. No.: US 2026/0191632 A1
(43) Pub. Date: Jul. 9, 2026

(54) Apparatus and Methods for Treating and Preventing Disease in Livestock

(71) Applicant: Centre for Dairy Intelligence Limited, Putaruru (NZ)

(72) Inventor: Leon Bryan Spurrell, Etobicoke (CA)

(21) Appl. No.: 19/134,334

(22) PCT Filed: Dec. 22, 2023
(86) PCT No.: PCT/IB2023/063159

§ 371 Date: May 30, 2025

(30) Foreign Application Priority Data
Dec. 22, 2022 (NZ) 795951

(51) Int. Cl. A61D 1/02; A61D 7/00; A61D 9/00; A61L 15/18; A61L 15/44; A61L 27/04

(52) U.S. Cl. CPC A61D 1/02; A61D 7/00; A61D 9/00; A61L 15/18; A61L 15/44; A61L 27/045; A61L 27/047

(57) ABSTRACT

The invention is a medicament apparatus (1) for use with animals comprising a body (10) made from one or more solid metals, the metals selected from among those for which their ions have biocidal effect. The body comprises an internal porous matrix (11) of at least 1% porosity from which biocidal metal ions can be released, and an external surface of the body (10) is configured to be situated at a treatment site (20) of an animal (2).

### FIELD OF THE INVENTION

The present invention relates to an apparatus and/or methods for treating and preventing disease in livestock animals. More particularly, but not exclusively, it relates to a medicament apparatus and/or method for treating and preventing disease in cattle.

### BACKGROUND OF THE INVENTION

For farmers keeping livestock, the health of the animals is a key concern. Lameness, mastitis and reproductive issues are the most common health problems for cattle, and they often lead to steep financial costs that significantly impacts on farm profits. Mastitis is most commonly caused by bacteria invading the udder, and lameness is sometimes caused by infections of and around the hoof.

Because of the considerable expense of identifying and treating such issues, preventative solutions that can be administered en masse may be most economical. Antibiotics are very widely used for this purpose. To prevent or treat hoof issues such as digital dermatitis or foot rot, antibiotics are sometimes used in conjunction with harsh chemical baths for the hooves. To prevent mastitis, antibiotics are used in conjunction with teat sealants typically applied by syringe. These syringes are typically single-dose, which creates high volumes of plastic waste at scale.

One major problem with the widespread use of antibiotics as disease preventatives in livestock is that it accelerates the development of antibiotic resistance in the targeted bacterial strains. This is gradually rendering common antibiotics ineffective, which will eventually leave farmers to contend with failing livestock health and no cost-effective and useful preventative medicine available. This problem is now beginning to impact the industry significantly, making it increasingly desirable to find alternative solutions for preventing and/or treating common health issues such as mastitis and lameness. Alternative solutions would need to still be effective against bacteria, while also being cost-effective and simple to manufacture and administer.

One avenue that has begun to be explored is the use of metals that have biocidal properties. Metals such as copper are known to kill bacteria and other microorganisms, which is generally understood to be a result of metal ions damaging the cell walls of the microorganisms.

There is some existing research into the use of aqueous solutions of copper for animal medicinal purposes, and U.S. Pat. No. 4,418,686 discloses an animal implant device that uses bands of metal to create a galvanic cell releasing ions for antibacterial effect. However, these existing attempts to capitalise on the antibacterial properties of such metals have shortcomings in that the amount of biocidal ions provided is not very high, meaning that they are a poor substitute to antibiotics which would be much more effective at least until resistance is eventually developed. Additionally, devices such as that shown in U.S. Pat. No. 4,418,686 have a high manufacturing complexity that would make them much more expensive and uneconomical compared to mass application of antibiotics. They also have a short lifecycle, and the components that are left over when the implant is depleted can cause injury to the treatment site.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

For the purpose of this specification, where method steps are described in sequence, the sequence does not necessarily mean that the steps are to be chronologically ordered in that sequence, unless there is no other logical manner of interpreting the sequence.

It is an object of the present invention to provide an apparatus and method which overcomes or at least partially ameliorates some of the abovementioned disadvantages or which at least provides the public with a useful choice.

### BRIEF DESCRIPTION OF THE INVENTION

According to a first aspect the invention broadly comprises a medicament apparatus for use with animals comprising:
a body made from one or more solid metals, the metals selected from among those for which their ions have biocidal effect;
wherein the body comprises an internal porous matrix of at least 1% porosity from which biocidal metal ions can be released, and
wherein an external surface of the body is configured to be situated at a treatment site of an animal.

According to another aspect the one or more solid metals are selected from the group consisting of copper, cobalt, zinc, nickel, zirconium, molybdenum, and alloys of the same.

According to another aspect the one or more solid metals are pure metals such that they are not alloys.

According to another aspect the body is made from only a single solid metal.

According to another aspect the body is made from pure copper.

According to another aspect the internal porous matrix has at least 10% porosity.

According to another aspect the internal porous matrix has between 20% to 80% porosity.

According to another aspect the internal porous matrix has an average pore size of 50 microns or less.

According to another aspect the body has one or more flow channels therethrough.

According to another aspect the invention broadly comprises an implant, and the body is no more than 20 mm in length in any dimension.

According to another aspect the body has a plurality of grooves in the external surface.

According to another aspect the body is a gyroid structure.

According to another aspect the internal porous matrix has at least 50% porosity.

An elongate plug suitable for insertion into a teat canal of a lactating animal, wherein the elongate plug is a medicament apparatus.

According to another aspect the elongate plug comprises a port at each of a proximal end and a distal end to facilitate fluid drainage in one direction and administration of medicines in the other direction.

According to another aspect the internal porous matrix has 20% porosity or less.

A patch suitable for application to an external treatment site of an animal, wherein the patch is a medicament apparatus and the body is formed as a thin layer.

According to another aspect the thin layer has a plurality of grooves in the external surface.

According to another aspect the thin layer presents or is formed as a crisscrossing lattice.

According to another aspect the thin layer presents one or more rough sections with high density of peaks.

According to another aspect the invention broadly comprises a method of manufacturing the medicament apparatus, wherein the method is an additive manufacturing process and comprises a step of metal 3D printing.

According to another aspect the additive manufacturing process is binder jetting of a metal powder.

According to another aspect the metal powder's granule size is 50 microns or less.

According to another aspect the metal powder's purity is at least 99.95%.

According to another aspect sintering cycles that complete the binder jetting process are of low intensity such that voids in the internal porous matrix are mostly retained.

According to another aspect prior to binder jetting a space holder material is mixed with the metal powder, the space holder material being subsequently removed by sintering thereby creating pores in the internal porous matrix.

According to another aspect the space holder material is selected from the group consisting of potassium carbonate, sodium chloride, and carbamide.

According to another aspect the space holder material is needlelike.

According to another aspect the invention broadly comprises a method of treating or preventing infection in livestock animals using the medicament apparatus, wherein the method comprises applying the medicament apparatus to a treatment site of the animal.

According to another aspect wherein the method comprises injecting the medicament apparatus into skin near a hoof of the animal to prevent or treat foot rot, digital dermatitis, inter-digital dermatitis, white line disease, and/or sole ulcers.

According to another aspect the method comprises inserting the medicament apparatus into a teat canal of the animal to prevent or treat mastitis and/or flystrike, to prevent contaminants from entering the teat canal, or as a cannula, teat straightener, internal teat canal skin conditioner, or teat end closure.

According to another aspect the method comprises externally adhering the medicament apparatus to a wound of the animal to prevent or treat infection.

According to another aspect the animal is a lactating animal selected from the group consisting of cow, goat, pig, deer, buffalo, sheep, and camel.

According to another aspect the invention broadly comprises a medicament apparatus for use with animals, the medicament apparatus comprising:
a mounting member;
one or more bodies each made from one or more solid metals, the metals selected from among those for which their ions have biocidal effect;
wherein the one or more bodies are mounted on the mounting member and an external surface of each of the bodies is configured to be situated at a treatment site of an animal.

According to another aspect the medicament apparatus is formed as an elongate plug suitable for insertion into a teat canal of a lactating animal.

According to another aspect the mounting member is a shaft, and the one or more bodies are mounted along the length of the shaft.

According to another aspect the shaft defines a widened region towards one end.

According to another aspect the widened region is defined by adjacent branches of the shaft which diverge and reconverge.

According to another aspect the medicament apparatus further comprises an end stop fixed at an opposite end of the shaft.

According to another aspect the one or more bodies comprise beads mounted on the shaft.

According to another aspect the one or more bodies comprise wire wound about the shaft.

According to another aspect the mounting member is flexible and resilient.

According to another aspect the mounting member is made from a shape memory alloy.

According to another aspect the mounting member is made from nitinol.

According to another aspect the one or more solid metals are selected from the group consisting of copper, cobalt, zinc, nickel, zirconium, molybdenum, and alloys of the same.

According to another aspect the one or more solid metals are pure metals such that they are not alloys.

According to another aspect each of the one or more bodies is made from only a single solid metal.

According to another aspect each of the bodies is made from pure copper.

Other aspects of the invention may become apparent from the following description which is given by way of example only and with reference to the accompanying drawings.

As used herein the term “and/or” means “and” or “or”, or both.

As used herein “(s)” following a noun means the plural and/or singular forms of the noun.

The term “comprising” as used in this specification and claims means “consisting at least in part of”. When interpreting statements in this specification and claims which include that term, the features, prefaced by that term in each statement, all need to be present but other features can also be present. Related terms such as “comprise” and “comprised” are to be interpreted in the same manner.

### BRIEF DESCRIPTION OF THE DRAWINGS

The invention will now be described by way of example only and with reference to the drawings in which:

FIG. 1 shows a cross-sectional closeup view of the internal porous matrix;

FIG. 2A shows a perspective view of a first embodiment of the implant;

FIG. 2B shows a perspective cross sectional view of the first embodiment of the implant where the internal flow channels are visible;

FIG. 3A shows a perspective view of a second embodiment of the implant;

FIG. 3B shows a side cross sectional view of the second embodiment of the implant;

FIG. 4 shows a perspective view of the third embodiment of the implant;

FIG. 5 shows a perspective view of a longer variant of the third embodiment of the implant;

FIG. 6 shows X-ray views of implants injected into a treatment site near a cow's hoof;

FIG. 7A shows a perspective view of a first embodiment of the plug;

FIG. 7B shows a top view of a first embodiment of the plug;

FIG. 8A shows a perspective view of a second embodiment of the plug;
shows a top view of the second embodiment of the plug; FIG. 9B

FIG. 8C shows a side cross-sectional view of the second embodiment of the plug;

FIG. 9 shows an X-ray view of the plug inserted into a cow's teat canal;

FIG. 10A shows a top perspective of a first embodiment of the patch;

FIG. 10B shows a bottom perspective view of the first embodiment of the patch;

FIG. 11A shows a top perspective view of a second embodiment of the patch;

FIG. 11B shows a bottom perspective view of the second embodiment of the patch;

FIG. 12 shows a top perspective view of the third embodiment of the patch;

FIG. 13A shows a top view of a third embodiment of the plug; and

FIG. 13B shows a top view of a fourth embodiment of the plug.

### DETAILED DESCRIPTION OF PREFERRED EMBODIMENTS

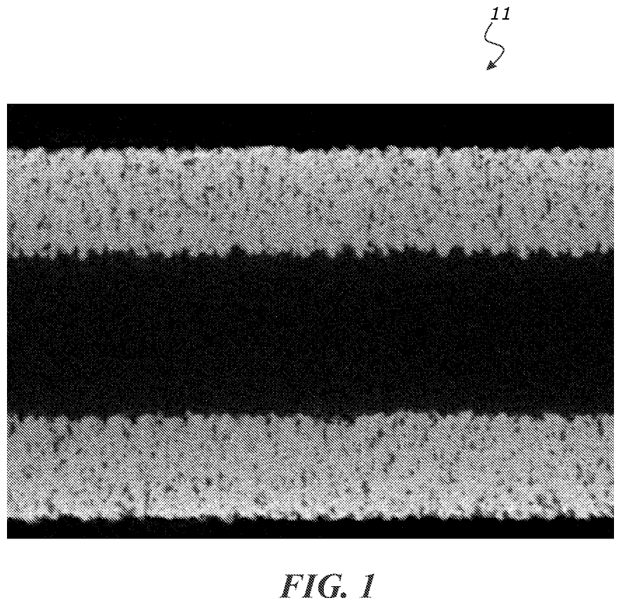

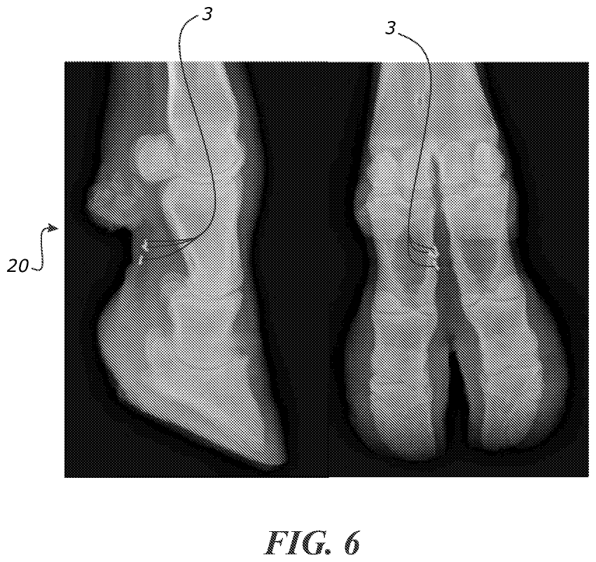

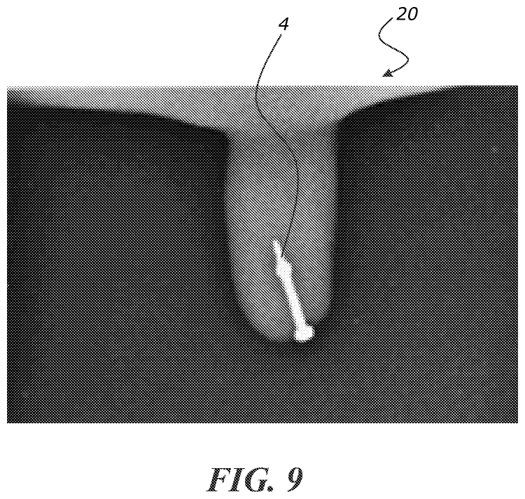

According to various aspects of the present invention as illustrated in FIGS. 1-9B, there is provided a medicament apparatus 1, a method of manufacture and method of treating and preventing disease in animals which will now be described.

It will be appreciated that these figures illustrate the general principles of the structure and construction, and that the invention is not limited to the precise configurations illustrated.

### General Structure and Composition

The medicament apparatus 1 comprises a body 10 made from one or more solid metals, the metals selected from among those for which their ions have biocidal effect. As shown in FIG. 1, the body 10 comprises an internal porous matrix 11 from which biocidal metal ions can be released. FIG. 1 is a close-up image of a cross section of a body 10 having an internal cavity, the upper and lower sections being walls of the body 10 comprising an internal porous matrix 11 of porous copper. Preferably the body 10 is unitary.

Biocidal effect is defined to mean that the ions are efficacious in killing or otherwise inactivating cells of harmful microorganisms such as bacteria, viruses, and parasites. This can be via surface contact of the organism with the metal, or the ions may be carried away from the metal, for example suspended in bodily fluids, and thereby affect harmful microorganisms that are not necessarily present on the metal surface.

An external surface of the body 10 is configured to be situated at a treatment site of an animal 2. This treatment site 20 could be an external treatment site, for example on a wound or another external site, or an internal treatment site, for example inside the teat canal or beneath the skin near the hoof. The medicament apparatus 1 might be placed at the treatment site 20 by hand or with the assistance of an appropriate instrument.

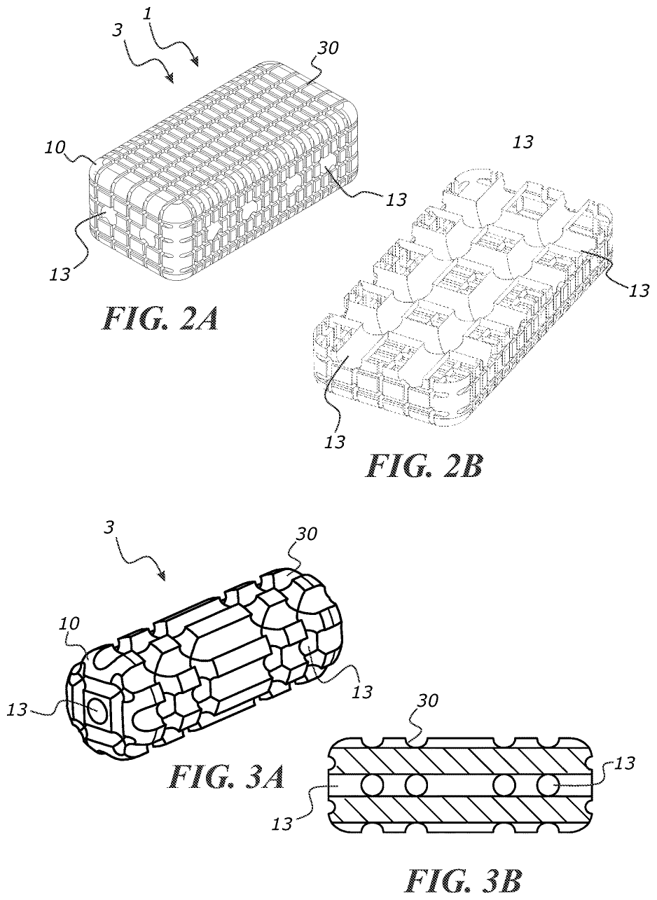

FIGS. 2-9B illustrate various embodiments of the medicament apparatus 1 suitable for various treatment sites 20, including variants of an implant 3, an elongate plug 4, and a patch 5. Each embodiment will be further described herein.

The terms “medicament” and “treatment site” are used in this specification in a broad sense so as to encompass uses such as prevention of infection, treatment of an active infection, and/or provision of other health benefits. The animal 2 receiving the medicament apparatus 1 is intended to be a livestock animal, and preferably a bovine animal i.e. cattle, but the present invention may be suitable for use with a wider variety of animals as will be described.

By providing a source of biocidal metal ions, the medicament apparatus 1 kills or at least inhibits invasive bacteria entering the treatment site 20, thus functioning as an infection preventative. It can also kill bacteria at an already infected treatment site 20, thus treating the infection to some extent. By functioning with a different mechanism to antibiotic drugs, the development of antibiotic resistance in bacteria strains is hindered. Additionally, the internal porous matrix 11 can optionally be filled with medicament or other useful substances such as silver, zinc, solids, liquids, gels, antibiotics, micronutrients, or growth hormones prior to application at the treatment site 20, which will be slowly released into the treatment site 20 over time. Such substances may also coat the exterior of the body 10. This can provide another mechanism for killing invasive bacteria or promoting healing.

For example, the internal porous matrix 11 may be filled with colloidal silver from 1-22,000 ppm as a secondary source of biocidal metal ions in addition to the metal of the internal porous matrix 11 itself.

Multiple metal elements are known to have biocidal properties, and in particular to have ions with biocidal properties. For the purposes of the present invention, the solid metal(s) used to form the body on account of such biocidal properties are preferably selected from the group of copper, zinc, silver, cobalt, nickel, zirconium, molybdenum, and alloys of the same. Some such metals may be less preferable than others, especially those which can have poisonous effects in low quantity such as lead.

Preferably the one or more solid metals are pure metals, rather than being alloys or another molecular compound. The term “pure metal” is intended only to mean that the metal is a chemical element, in contrast to an alloy or molecular compound, and it does not exclude the possibility that other compounds might be present in negligible amounts within the metal without constituting an alloy. Compounds that might be present in small amounts could include residue from liquid binding agents used in the manufacturing process, or small impurities in metal powder used in the process. A metal with 99.95% purity would for example be considered “pure” for the purposes of this specification.

Preferably, the body 10 is made from only a single solid (and preferably pure) metal to simplify the manufacturing process, reduce cost, and provide better control over biocidal ion release. However, if the body 10 is made from multiple solid metals (pure or alloy), they could have an interface between them such that the body 10 is split into multiple sections. The interface may be a mechanical connection such as a press fit, or the dissimilar sections could be fused together by some other manufacturing process.

A preferable metal for the body 10 is pure copper, as copper ions have particularly strong biocidal effect, copper is widely known and approved as a biocidal, and copper is a micronutrient for most animals. Copper is also widely available and suitable for various manufacturing processes, as it is easily shaped and formed, has a long shelf-life, and is highly recyclable without damaging or affecting it. Because silver and zinc ions also have particularly strong biocidal effect, the next most preferable metals are pure zinc, pure silver, and any combination of 2-3 of copper, zinc, and silver.

Release of the biocidal metal ions is facilitated by the internal porous matrix 11, which provides an abundance of sites for ion release due to high internal surface area. These ions will naturally be released into the treatment site gradually, in effect creating a slow-release mechanism. The internal porous matrix 11 also allows bodily fluids such as water, blood or mucus in the treatment site to enter the pores and stimulate the release of ions due to the presence of aqueous electrolytes. The rate of the ion release is influenced by the properties of the internal porous matrix 11, namely its porosity/density, the dimensions of the pores, and other such factors. In general, when the internal porous matrix 11 has a greater internal surface area, the rate of ion release is increased.

Increased porosity of the internal porous matrix 11 increases the internal surface area, thus it is generally desirable to maximise the porosity. Porosity can be expressed as a percentage representing the proportion of volume made up by voids in the material. It is related to the bulk density or effective density of the material, which is a measure of material density that includes void/pore volume. Higher porosity corresponds to lower bulk density.

The internal porous matrix 11 has a porosity of at least 1%, and preferably has a porosity of at least 10%. Matrix porosities of 20% and 30% are known to the applicant to be of reasonably good structural integrity based on experiment, however porosities as high as 67% are known to be possible without being too fragile for some applications.

Porosity maximisation is largely a challenge of manufacturing, and preferred methods for creating high porosity metals will be described. Care must be taken to ensure that the structural integrity of the metal is retained. It will be appreciated that optimisation of manufacturing techniques and parameters can allow for even higher porosities than 67%, for example porosities as high as 80% may be achievable with the use of space holder materials as will be described. The present invention provides a general principle of application such that it is not limited to any particular maximum porosity except for the limit of what is physically possible.

Surface roughness of the external surface of the body 10 is another property that can affect the external surface area, and hence the rate of ion release. In general, higher surface roughness results in higher rates of ion release and is hence desirable. Surface roughness may also be correlated with the porosity of the internal porous matrix 11.

The internal porous matrix preferably has a pore dimension at the micrometre scale, for example average pore sizes of approximately 50 microns. The pore size will depend on the type of metal used and the method of manufacture as will be described. In general, smaller pore sizes increase the internal surface area, thus it is desirable to minimise the pore size.

One potentially limiting factor on the desired rate of ion release is metal toxicity considerations for the target animal 2. Regardless of the metal used for the body 10, an excess of metal ion release may have toxic effects on the animal 2 and cause health complications, thus defeating the intended purpose of the medicament apparatus 1. For this reason, the internal porous matrix 11 is preferably designed to release metal ions at a rate well below what could be toxic to a target animal 2. The medicament apparatus 1 is also preferably designed such that even if the entire structure was rapidly released into the animal 2, it would not become toxic.

The medicament apparatus 1 may be configured for a particular animal or selection of animals, for example the medicament apparatus 1 may have an internal porous matrix 11 with higher porosity if it is intended for use with cattle, but a lower porosity if intended for use with a smaller animal such as a sheep.

### Method

Following the description of the structure of the present invention described above, a general description of the method of forming the apparatus for treating and preventing disease in cows will now be described.

The medicament apparatus 1 is preferably manufactured by a metal additive manufacturing process, i.e. one involving a step of metal 3D printing. This allows the body and internal porous matrix 11 to be precision-manufactured with customised density/porosity and surface roughness properties that achieve the desired rate of biocidal ion release, and for the body 10 to be created in any desired form factor with appropriate flow channels 13. The specific additive manufacturing process used is preferably binder jet printing (i.e. binder jetting) which will be briefly described.

Other manufacturing processes may be suitable for producing medicament apparatus 1, provided that the process can produce porous metal structures. For example, CNC tooling could be used to manufacture pores in a subtractive way, but sufficiently accurate control would be more difficult and porosity customisation would be more limited compared to additive manufacturing processes herein described. Moulds could also be used with space holder material to cast porous metal structures, however it would be difficult to achieve internal features such as flow channels 13 at small scale. Mass production may also be more difficult.

In the preferred manufacturing process of binder jet printing, a roller deposits a layer of metal powder particles on to a print bed. An inkjet printhead then deposits a liquid binding agent on the layer to bind the particles. The print bed can then be lowered (or the relative height otherwise adjusted) and the process can be repeated multiple times, with the roller depositing another layer and the printhead binding it. A batch of parts may be printed and undergo the subsequent steps simultaneously.

Once the print is complete, a depowdering step is preferably performed to evacuate unbound metal powder from the part utilising compressed air and/or under specific controlled vacuum. Internal geometry of the medicament apparatus 1 is configured to ensure that unbound metal powder can be fully evacuated. Depowdering is preferably automated, but could potentially be performed manually.

Next, the binder may be cured by heating to give the part enough strength to allow handling.

Finally, the part undergoes one or more sintering cycles which remove the binding agent and densify the part into its final configuration, further improving its strength. Preferably the part is placed on a ceramic tray during sintering. Sintering cycles can be customised to achieve the desired porosity/density, surface roughness, or other properties of the part. The resulting solid metal component is then ready for is end-use application.

The selection of the metal powder used influences factors such as the pore dimension and internal surface area of the internal porous matrix 11. The pore dimension will largely be determined by the grain size of the powder, for example a 50 micron powder creates pores of a similar scale. Internal surface area increases with smaller granule size. Thus, it is generally desirable to select powders with small granule sizes.

One of several other factors that influence the porosity of the internal porous matrix 11 is the selection of the liquid binding agents. Various binder compositions are possible, selection occurring in combination with selection of an appropriate sintering cycle.

Prior to sintering, liquid binding agent is present in the metal which creates voids as it is removed from the structure during sintering. Thus, high porosity can be promoted by selection of appropriate low-intensity sintering cycles that do not overly densify the metal. This is contrary to the conventional objective of sintering, where typically strength is the primary consideration and maximised densification (i.e. removal of voids) is desirable. For the present invention, the objective of sintering is to remove the liquid binding agent but only densify the material to the minimum extent needed for the part to have sufficient structural rigidity. Thus, the voids will mostly remain and result in high porosity.

Optionally, a space holder material may be interspersed in the part prior to sintering (in addition to the liquid binding agent) to further promote increased porosity. The space holder may be a material such as potassium carbonate, sodium chloride, or carbamide, preferably in a needlelike form. The space holder can be mixed in with the metal powder prior to binder jetting, and subsequently removed from the part during sintering by means of a sintering cycle at an appropriate temperature. The shape, size, and fraction of space holder used can assist in customising the pore shape and dimensions as well as allowing for higher porosities than with a liquid binding agent alone.

The applicant has found that copper is well-suited to binder jet printing and sintering for high porosity, so in combination with its desirable biocidal properties it is the preferred metal for use with the additive manufacturing processes described for the present invention.

A preferred metal powder for use in the manufacturing process is approximately 50 micron copper powder or smaller. Preferably it also has at least 99.95% purity, and more preferably about 99.99% purity.

Additive manufacturing processes are relatively low-cost, low energy consumption, and non-polluting, which are advantages over alternative manufacturing methods. The metal used to manufacture each medicament apparatus can also be recycled into further products at end-of-life, as it can be converted back into powder by an appropriate smelting/melting or grinding process.

### Implant

As shown in FIGS. 2A-4, in some embodiments of the invention the body 10 is formed as an implant 3 suitable for use at an internal treatment site 20. It could be implanted beneath the skin by an implant syringe, implant gun, or any other suitable device that injects mechanically, electrically, or pneumatically. Preferably, the intended treatment site 20 is in the vicinity of the hoof of the target animal 2, such that the implant can prevent or treat infections around the hoof/foot of the animal 2.

When formed as an implant 3 for insertion beneath the skin, the body 10 is preferably of an appropriately small size to facilitate easy insertion. Preferably the body 10 is no more than 20 mm in length in any dimension, i.e. it could fit within a bounding sphere of 20 mm diameter. More preferably it is no more than 10 mm in any dimension.

In a first embodiment of the implant 3 as shown in FIGS. 2A-2B, it is formed as a cuboid with rounded edges. Preferably it has dimensions of approximately 6 mm long, 3 mm wide and 2 mm high.

In a second embodiment of the implant 3 as shown in FIGS. 3A-3B, it is formed as a cylinder with rounded edges. Preferably it has a length of approximately 6 mm and diameter of approximately 2.2 mm.

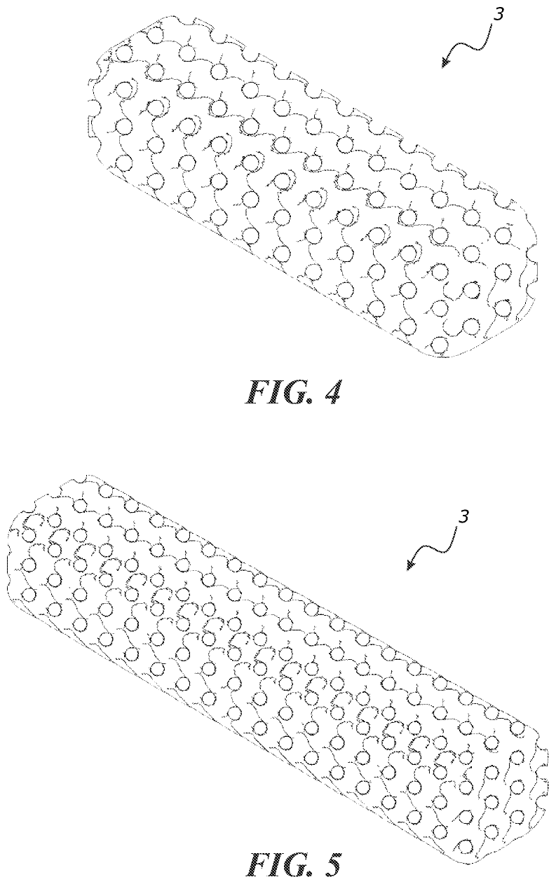

These dimensions allow the implants 3 to be injected via a needle, e.g. of a syringe or gun. Larger dimensions may still be injectable but would require larger needles that would be less comfortable for the animal, so it is preferable to inject multiple implants 3 to achieve the desired effect rather than using a larger implant. FIG. 4 shows multiple implants 3 injected below the skin near the hoof.

For the implant 3, the external surface of the body 10 preferably has grooves 30 so as to increase the external surface area and thereby increase the release rate of biocidal metal ions. The grooves 30 may also assist with retention in the internal treatment site, allowing tissue to grow around into the grooves 30 and secure the body 10.

The body 10 may also have one or more flow channels 13 therethrough to permit the passage of bodily fluids. This can ensure that flows of excess tissue fluid in the treatment site are not blocked, while also using the passage of the fluid to further activate the internal porous matrix and increase the rate of biocidal metal ion release. Tissue may also grow into the flow channels 13 to better secure the implant 3 in place, and the flow channels 13 may act as traps for bacteria.

In the first embodiment as shown in FIGS. 2A-2B, two flow channels 13 pass through the body 10 longitudinally and four flow channels 13 pass through the body horizontally. In the second embodiment as shown in FIGS. 3A-3B, one flow channel 13 passes longitudinally through the body 10 and four flow channels 13 pass through horizontally.

In a third embodiment of the implant 3 as shown in FIG. 4, the body 10 has a gyroid structure and the flow channels 13 are part of the gyroid. FIG. 5 shows a lengthened variant of the third embodiment. The gyroid structure has high strength, high surface area, and is easily 3D printed as part of the preferred manufacturing process.

For implants 3, provision of growth hormones, other metal ions/colloids, and/or micronutrients within the internal porous matrix 11 may be especially beneficial because slow release of such substances can promote growth and hardening of hoof keratin, thereby improving hoof thickness and formation. This could be especially useful when treating an existing hoof condition, as it could speed up recovery for example if the existing condition has caused the hoof to be partially decayed or malformed.

Copper itself is also an essential micronutrient for livestock animals such as cows and is typically provided as a nutritional supplement added into the feed for the animal. Implants 3 made of copper can thus provide some or all of the nutritional copper intake for the animal 2 by gradually releasing copper ions directly into the treatment site 20 via the internal porous matrix 11. Implants 3 can be inserted in a variety of sensitive locations on the cow to ensure that sufficient copper is absorbed, aiding in replacing nutritional supplements added to the feed.

The implant 3 can inactivate invasive bacteria and other microorganisms by means of the internal porous matrix 11. For example, when an implant 3 is injected near the hoof of the animal 2 it can act as a preventative for white line disease, foot rot, sole ulcers and other conditions which are typically caused by bacterial infection.

Over time, implants 3 will fully dissolve in the treatment site 20 due to the small dimensions of the implant 3 and the gradual release of material in the form of biocidal metal ions. Factors such as the size and porosity of the implant 3 will affect the time taken for it to fully dissolve, which can be monitored via X-ray scans to determine a suitable reapplication interval for a given embodiment of implant 3. For example, X-ray monitoring of the dissolution may indicate that for a particular embodiment of implants 3, annual reapplication is appropriate.

Because of the small scale of the implant 3 and the expectation that it will dissolve over time, high structural integrity after insertion into the treatment site 20 is not essential. Thus, especially high porosity of the internal porous matrix 11 is desirable for the implant 3 provided that it retains the minimum strength needed to be injected into the treatment site 20. For example, 50% or higher porosity may be suitable for the implant 3, when this level of porosity may be unsuitable for other applications.

Use of the present invention in the form of an implant 3 near the hoof has numerous advantages over existing hoof care solutions. Currently, it is common to regularly apply footbaths of harsh chemicals to prevent or treat hoof issues with livestock. These chemicals can be hazardous and must be transported and handled with caution.

In contrast, an implant 3 according to the present invention is non-toxic and can be safely handled and transported without any associated hazards. Implants 3 according to the present invention will also require less frequent application, for instance an annual application can substitute for the 2-7 footbaths annually with chemical solutions. Waste is also considerably reduced given that standard chemicals are only single-use.

Use of the implant 3 can also reduce the need for antibiotics as a preventative for the aforementioned hoof conditions, thus hindering the development of antibiotic resistance and also reducing or eliminating the withholding time on the sale of the animal or its milk product arising from the need to wait for antibiotics to pass through the system.

### Plug

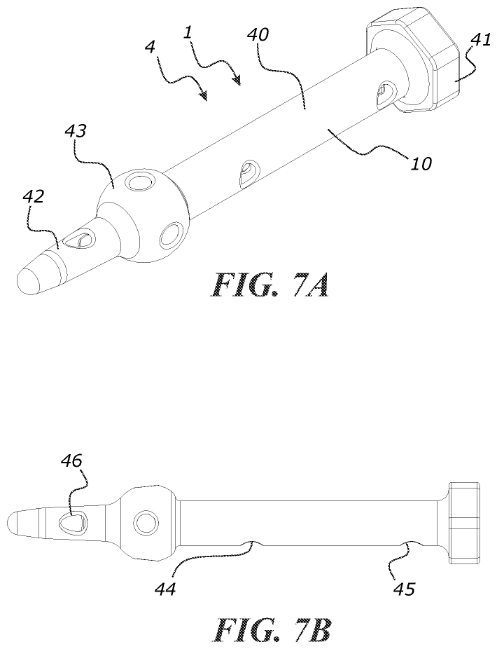

As shown in FIGS. 7A-9, in some embodiments of the invention the body 10 is formed as an elongate plug 4 suitable for insertion into a teat canal of a lactating animal. The lactating animal is preferably a cow, but could also be a goat, pig, deer, buffalo, sheep, camel, or any other animal that lactates.

In this configuration, the body 10 has features which are the same or similar to the ones described above in the first embodiment (the implant), for example the flow channels 13. Similar features are therefore generally indicated by the same reference numerals.

A primary difference in these embodiments is that the body 10 is shaped and sized such that it can be effective as a plug 4 for a teat. This preferably means a shaft 40 is provided with a roughly circular cross-section. For use with a cow, the shaft 40 preferably has a diameter of approximately 2 mm.

In a first embodiment of the plug 4 as shown in FIGS. 7A-7B, the plug 4 is approximately 20 mm long. This dimension is suitable for use with a cow teat.

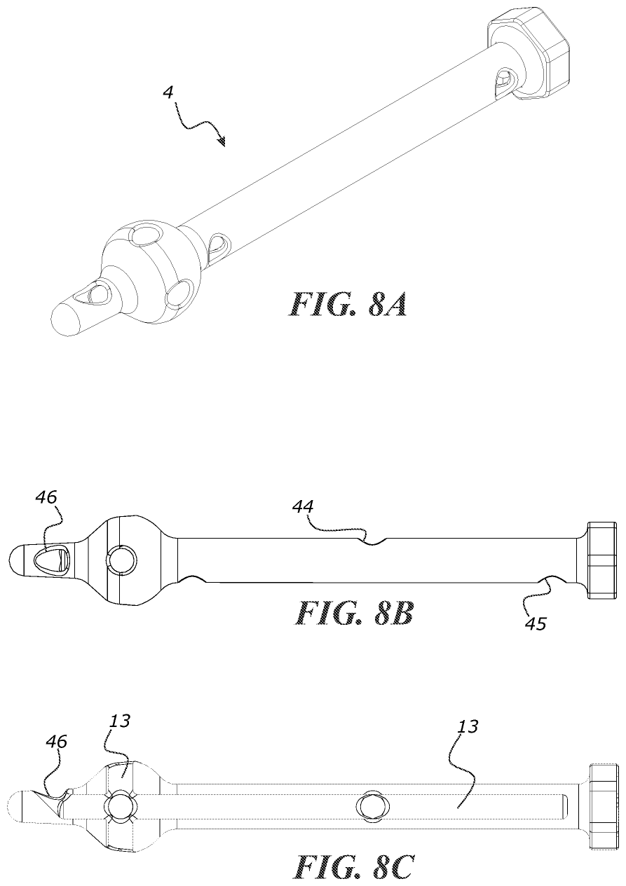

In a second embodiment of the plug 4 as shown in FIGS. 8A-8C, the plug 4 is approximately 27 mm long. This dimension is also suitable for use with a cow teat.

It will be appreciated that many variations of length and size are possible, however such variations must be in keeping with the size of the teat of the target animal 2. For example, a plug 4 for a sheep may be smaller than one intended for a cow. For a cow, the size range is preferably between 15-40 mm.

One flanged end 41 may act as an end stop, while the other end may be a tapered end 42 to facilitate easy insertion. The flanged end 41 can sit outside the teat and allow for easy removal, as well as acting as a barrier to dirt and bacteria.

For the plug 4, a longitudinal flow channel 13 can facilitate the drainage of excess milk in the udder through the teat canal via the plug 4, thereby relieving pressure and reducing infection risk. The longitudinal flow channel 13 can also be used in the opposite direction to administer medications deep into the teat and udder, for example if the teat or udder are damaged. The plug 4 preferably has a proximal end port 45 and a distal end port 46 to facilitate these functions.

Additional, intermediate ports 44 to the longitudinal flow channel 13 may be provided in the shaft 40. For example, in the first embodiment as shown in FIG. 7B there is one intermediate port 44 provided, while in the second embodiment as shown in FIG. 8B there are two intermediate ports 44 provided, one roughly in the middle of the shaft 40 and one towards the end.

As shown in FIGS. 7A-8C, the tapered end 42 preferably has a bulb 43 which can sit just inside the teat cistern to help retain the plug 4 in the teat canal. The bulb 43 may be pear-shaped, spherical, or another similar shape. The bulb 43 can contain additional horizontal flow channels 13 therethrough to allow the bulb 43 to act as a collection point for milk and other fluids, assisting with drainage. The bulb 43 can also act as a bacteria trap where more biocidal ions are released. As shown in FIG. 8C, the flow channels 13 in the bulb 42 are preferably in a cross shape. The distal end port 46 is preferably provided further along the tapered end 42 than the bulb 43.

The plug 4 may be installable in the teat canal by hand, or an appropriate tool may be provided which pushes the plug 4 into the teat canal. The plug 4 could be coated in a suitable lubricant to facilitate easy installation.

When installed in the teat canal, the plug 4 prevents invasive bacteria entering the teat canal by means of the internal porous matrix 11, thus preventing the development of mastitis in the animal (i.e. bovine mastitis for cows). It can also act to prevent parasitic diseases that can afflict the teats, for example it can prevent flystrike by denying entry to blowflies and maggots.

The plug 4 can thus be installed in the early weeks of the dry period when the animal 2 is not producing milk, which is typically when mastitis infections are prone to developing. However, even in the lactation period the plug 4 could also be installed between milking sessions to prevent infection or flystrike, which may be especially beneficial for cows that are leaking milk. Plugs 4 can be removed prior to the calving period, after approximately 60 days of application, before reapplication at the start of the next cycle.

Because the plug 4 is intended to remain blocking the teat canal for an extended period, it is important that it has enough structural integrity that it will not dissolve or become damaged/deformed too early or easily. Thus, a lower level of porosity for the internal porous matrix 11 is preferable compared to an application such as implant 3. For example, a porosity in the vicinity of 20% may be suitable for plug 4 as a balance between high release rate and structural strength.

The plug 4 can also have other benefits that arise from ion release conditioning the skin in the teat canal, which prevents drying, cracking, and deformation of the teat. Because it is in effect self-decontaminating, the plug 4 can be used as a tool during surgery or treatment of damaged teats, teat cisterns or teat canals.

The plug 4 acts as a replacement for teat sealants that are typically administered applied by syringe during the dry period to prevent mastitis. However, the plug 4 is simpler to remove and thus is more suitable for use between milking during the lactation period. It is simpler and less wasteful to install because it does not require pre-filled single-use syringes, and rather any installation tool would be reusable. Additionally, the plug 4 itself could also be recycled, or cleaned and reused.

### Patch

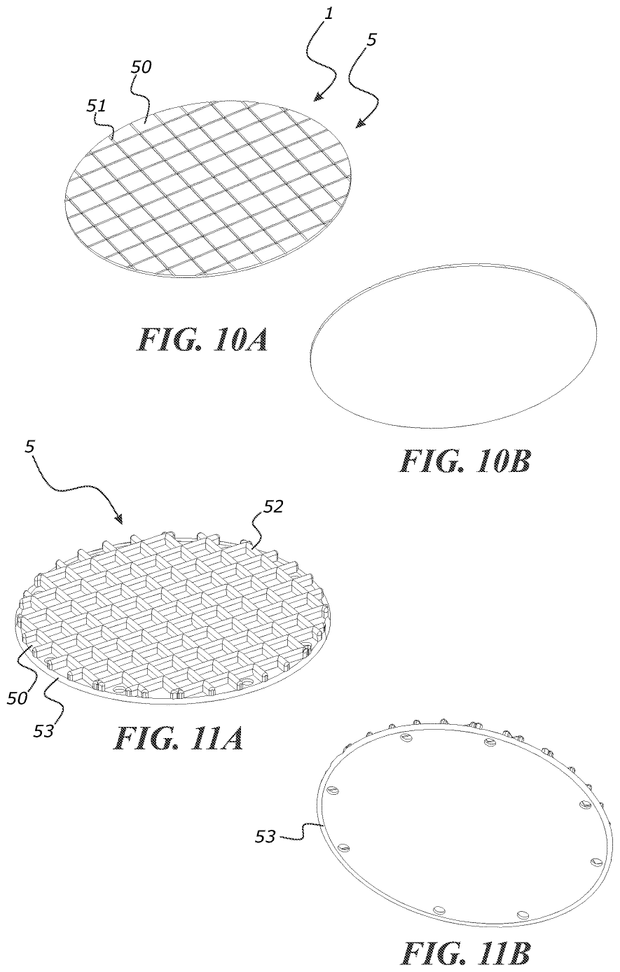

As shown in FIGS. 10A-10B, in some embodiments of the invention the body 10 is formed as a thin layer 50 that can be applied to an external treatment site as a patch 5. This embodiment may be particularly suited for treating wounds or existing infections by direct application to the afflicted treatment site.

The patch 5 could be circular, square, rectangular, or any necessary shape to fit specific applications, and has a standard diameter of approximately 5 mm. However, it will be appreciated that many variations in size and shape are possible, for example the patch 5 could be some other polygonal shape. It could be made in various sizes, for example it could be up to 100 mm in diameter to cover larger wounds.

In a first embodiment of the patch 5 as shown in FIGS. 10A-10B, the thin layer 50 has a plurality of grooves 51 formed in it. The grooves 51 contribute to increased external surface area, and may also assist with retention at the treatment site 20 by allowing tissue to grow into the grooves 51. Preferably the grooves 51 are in a crisscrossing pattern.

In a second embodiment of the patch 5 as shown in FIGS. 11A-11B, the thin layer 50 presents a crisscrossing lattice 52 which contributes to high mechanical stability under pressure. The thin layer 50 may have an annular rim 52 to provide support to the lattice 52. The lattice 52 may itself form the thin layer 50, such that that the lattice 52 is supported solely by the rim 52.

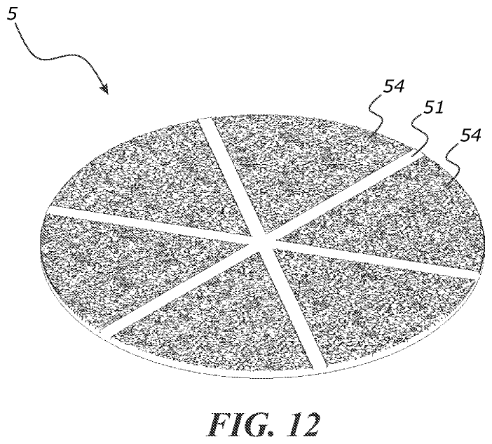

In a third embodiment of the patch 5 as shown in FIG. 12, on one side the thin layer 50 presents a plurality of rough sections 54 having high peak density. The rough sections 54 are separated by radial grooves 51 having a smoother surface. However, the rough sections 54 could also be separated in some other manner, or there may be one rough section 54 that spans some or all of the thin layer 50. The rough sections 54 have especially high surface area which is beneficial for biocidial ion release, and tissue growth can also occur into the peaks to better retain the patch 5 in place.

Preferably, the patch 5 is flexible (i.e. can easily deform elastically and/or plastically) and can conform to the contour of the external treatment site. It may be secured with an adhesive of some kind, for example a glue or tape, or plastic deformation of the patch may be sufficient to retain it in place—for example it could be wrapped around the leg or the hoof and kept in place by the rigidity of the metal.

The thickness of the patch 5 is preferably at either the millimetre or micron scale but may depend on the structure of the thin layer 50. For example, when the lattice 52 forms the thin layer 50 it is preferable for the thin layer 50 to have a higher thickness of a small number of millimetres. If the thin layer 50 is formed with a more solid structure rather than as a lattice 52, then smaller thicknesses in the micron range could be suitable.

In the patch 5, the internal porous matrix 11 may be filled with micronutrients or growth hormones to stimulate the growth of tissue to close the underlying wound. Although it can function independently, the patch 5 could also be used in conjunction with other treatment aids to speed up the healing process.

The patch 5 inactivates bacteria and other microorganisms in the treatment site 20 by means of the internal porous matrix 11 and can thus act as an alternative to harsh chemicals that are sometimes used to treat external infections. Another benefit of the patch is that it leaves no residue. Because of the small, thin nature of the patch 5 and its intended use as a treatment aid, it is suited to use as a single-use disposable and recyclable aid.

When manufactured with additive manufacturing processes such as binder jetting, the thin layer 50 can be constructed in a relatively small number of passes of the roller. This makes the patch 5 particularly suited to rapid mass production.

### Compound Apparatus

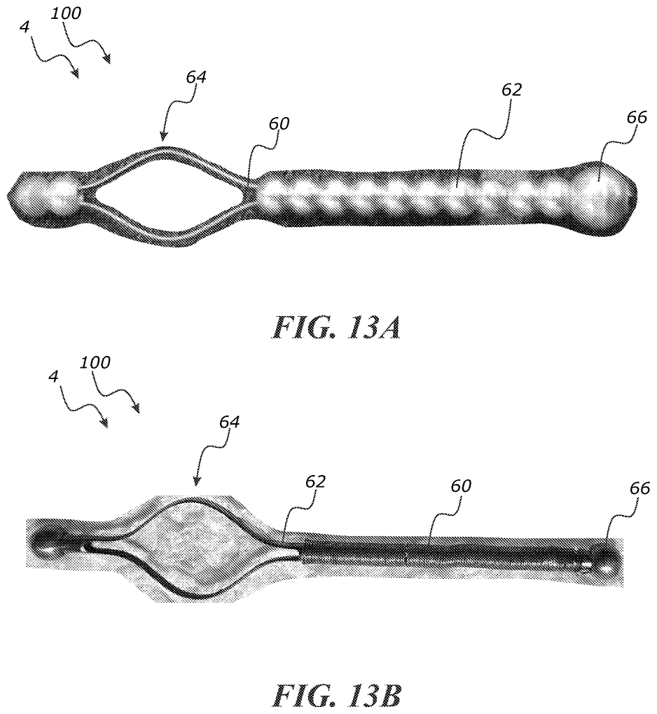

As shown in FIGS. 13A-13B, in another embodiment the medical apparatus is not made up of a singular unitary body 10. Rather, the medical apparatus is a ‘compound’ medical apparatus 100 which comprises multiple parts assembled together.

The compound medical apparatus 100 comprises a mounting member 60 and one or more bodies 62 mounted on the mounting member 60. The bodies 62 are made from one or more solid metals selected from among those for which their ions have biocidal effect, in the same way as previously described. An external surface of each of the bodies 62 is configured to be situated at a treatment site of an animal 2.

Preferably, the compound medical apparatus 100 is formed as an elongate plug 4 suitable for insertion into a teat canal of a lactating animal. Thus, FIGS. 13A and 13B respectively provide for third and fourth embodiments of the elongate plug 4 as previously described. The size for the third and fourth embodiments is preferably similar to the previous embodiments of the elongate plug 4, for example between 15-40 mm in length.

The mounting member 60 is preferably a shaft to provide the basic shape of the elongate plug 4, although the shaft may be more complex in shape than a simple axial extent. The one or more bodies 62 are mounted along the length of the shaft, although not necessarily along the entire length.

In the third embodiment as shown in FIG. 13A, at least some of the one or more bodies 62 are formed as beads, which may be slidably moveable along the shaft to some extent. Some of the bodies 62 may be similar in shape but fixed in place, for example at either end of the shaft to act as end stops which prevent the slidably moveable bodies 62 from coming free from the shaft. The end stops could alternatively be formed in the shaft itself, or be made of some other material from the bodies 62.

In the fourth embodiment as shown in FIG. 14B, the one or more bodies 62 are formed as wire wound around the shaft as shown in FIG. 13B. The wire could be provided pre-wound and slid over the shaft during manufacture, e.g. as a coil spring, or wound about the shaft during manufacture.

The one or more bodies 62 may be manufactured in an equivalent way as the body of previously described embodiments, for example by additive manufacturing which creates an internal porous matrix 11. However, the one or more bodies 62 may also be made by other methods (e.g. conventional machining) and may not necessarily comprise an internal porous matrix 11. Because the mounting member 60 is present to provide structural strength, the shape of the one or more bodies 62 can provide for increased surface area (which might otherwise be achieved by porosity) without compromising the structure of the medicament apparatus 100.

In the third embodiment, the bodies 62 formed as beads have a through-hole which increases the available surface area for biocidal ion release, especially when loosely fit such that the beads are slidably moveable along the shaft. In the fourth embodiment, formation as a thin wire inherently increases exposed surface area for a given amount of material, in comparison to a solid body such as a cylinder.

Preferably, the shaft defines a widened region 64 towards one end. The widened region 64 serves an equivalent purpose to the bulb 43 of previous embodiments, in that it may help retain the elongate plug 4 in the teat canal while still allowing drainage. The widened region 64 is preferably defined by adjacent branches of the shaft which diverge and reconverge, which is especially efficient in terms of material usage. Preferably, the one or more bodies 62 are not present in the widened region 64, and are only mounted on one or both sides of the widened region 64.

Preferably, the mounting member 60 is flexible and resilient. This allows the widened region 64 to contract and expand at least to some extent. The use of beads or wire for the one or more bodies 62 also facilitate flexing of the elongate plug 4. If the animal 2 lays on its teats, flexing of the elongate plug 4 can prevent internal injury or bruising. It also allows for easier insertion of the elongate plug 4 by hand without the use of any specialised instrument, and reduces fragility.

Preferably, the material of the mounting member 60 is inert, non-corrosive, and resistant to bacteria growth, but is a different material from the one or more bodies 62.

The mounting member 60 may be made from a shape memory alloy, preferably nitinol (i.e. nickel-titanium), which provides a sufficient degree of resilience as well as strength. Utilising the material properties of shape memory alloys, the mounting member 60 may be taught to ‘memorise’ the desired shape during manufacture by means of an appropriate thermal treatment. This allows for the widened region 64 to be formed by flexing the adjacent branches of the shaft, and subsequently fixating the resulting geometry such that it becomes the at-rest state of the mounting member 60. The shape memory is preferably configured to be retained for a high number of flex cycles, for example one million or more, such that the desired shape will be retained through an extended period of insertion (e.g. 2 months, which is the typical dry period for a cow).

Preferably, the compound medicament apparatus 100 further comprises an end stop 66 fixed at an opposite end of the shaft to the widened region 64. The end stop 66 may for example be spherical or pear-shaped, and may itself be one of the bodies 62 made from biocidal ion-releasing metal(s). It may be enlarged in comparison to the beads. However, various other shapes for the end stop 66 are possible. The end stop 66 can sit just outside the teat and act as a barrier to bacteria, equivalent to the flanged end 41 of previous embodiments.

As with previous embodiments, the one or more bodies 62 may optionally be filled/coated with medicament or other useful substances prior to application at the treatment site 20. Such substances may also coat the mounting member 60. This can provide another mechanism for killing invasive bacteria or promoting healing.

### SUMMARY

The medicament apparatus 1 achieves high rates of biocidal metal ion release into a treatment site 20 of a target animal 2 by provision of an internal porous matrix 11 in a body 10 made of a suitable metal. The rate of release will be increased with higher porosity and lower pore dimension because these factors increase the internal surface area from which ions can be released.

Additive manufacturing, and especially binder jet printing, can be used to create a body 10 with an internal porous matrix 11 by binding together metal powder particles. Various steps can be taken to promote high levels or porosity and small pore dimension, for example using small granules, using a liquid binding agent that creates many voids, and using a sintering cycle of low intensity to avoid closing too many voids.

The medicament apparatus 1 can be formed in various embodiments, for example the implant 3, plug 4 and patch 5 as have been described. Other medicament apparatus 1, for example ear tags, could equally be made in the same manner to benefit from release of biocidal ions. It is also possible that other devices could be made in whole or in part in the same manner, for example surgical equipment, milking cup liners, or water supply filters.

The applicant has conducted experiments to test the rate of biocidal metal ion release for medicament apparatus 1 according to the present invention.

In a first experiment, medicament apparatus 1 in the form of plugs 4 were submerged in samples of water for multiple days, after which the volume of residual copper in the water sample was measured. The results are summarised as follows.

For a control sample of distilled water, the residual copper level was 0.6 micrograms per litre after 11 days.

For a sample containing eleven plugs 4 of low porosity submerged for 9 days, the residual copper level was 3130 micrograms per litre.

For a sample containing four plugs 4 of lower porosity submerged for 9 days, the residual copper level was 958 micrograms per litre.

For a sample containing one plug 4 of high porosity submerged for 6 days, the residual copper level was 2380 micrograms per litre.

As is clear from the results, increased porosity of the plug 4 greatly increases the rate of ion release and hence the efficacy when applied to a treatment site 20 on an animal 2.

In a second experiment, medicament apparatus 1 in the form of implants 3 were subjected to the same experiment. The results are summarised as follows.

For a control sample of distilled water, the residual copper level was 10.7 micrograms per litre after 7 days.

For a sample containing one implant 3 of low porosity submerged for 7 days, the residual copper level was 86.9 micrograms per litre.

For a sample containing two implants 3 of low porosity submerged for 7 days, the residual copper level was 151 micrograms per litre.

For a sample containing three implants 3 of low porosity submerged for 7 days, the residual copper level was 205 micrograms per litre.

For a sample containing four implants 3 of low porosity submerged for 7 days, the residual copper level was 261 micrograms per litre.

For a sample containing five implants 3 of low porosity submerged for 7 days, the residual copper level was 327 micrograms per litre.

As is clear from these results, the use of multiple implants 3 can increase the rate of biocidal ion release within a treatment site 20, but not necessarily in a linear fashion.

The compound medicament apparatus 100 can likewise achieve high rates of biocidal ion release from the one or more bodies 62 into a treatment site 20, and is especially suited for use as an elongate plug 4. It also provides for flexibility which can prevent injury to the animal 2.

To those skilled in the art to which the invention relates, many changes in construction and widely differing embodiments and applications of the invention will suggest themselves without departing from the scope of the invention as defined in the appended claims.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

1. A medicament apparatus for use with animals comprising:
a body made from one or more solid metals, the metals selected from among those for which their ions have biocidal effect;
wherein the body comprises an internal porous matrix of at least 1% porosity from which biocidal metal ions can be released, and
wherein an external surface of the body is configured to be situated at a treatment site of an animal.

2. The medicament apparatus of claim 1, wherein the one or more solid metals comprises one or more pure solid metals selected from the group consisting of copper, cobalt, zinc, nickel, zirconium, molybdenum, and are not alloys.

3. (canceled)

4. (canceled)

5. The medicament apparatus of claim 2, wherein the body is made from pure copper.

6. (canceled)

7. The medicament apparatus of claim 1, wherein the internal porous matrix has between 20% to 80% porosity.

8. (canceled)

9. The medicament apparatus of claim 1, wherein the body has one or more flow channels therethrough.

10. The medicament apparatus of claim 1, wherein the medicament apparatus is formed as one of:
(a) an implant suitable for use at an internal treatment site of an animal, and the body is no more than 20 mm in length in any dimension and the body is a gyroid structure;
(b) an elongate plug suitable for insertion into a teat canal of a lactating animal, and the elongate plug comprises a port at each of a proximal end and a distal end to facilitate fluid drainage in one direction and administration of medicines in the other direction; or
(c) as a patch suitable for application to an external treatment site of an animal, the body is formed as a thin layer, and the thin layer has a plurality of grooves in the external surface.

11-19. (canceled)

20. The medicament apparatus of claim 10, wherein the medicament apparatus as formed as the patch comprises one or more rough sections with high density of peaks on the thin layer.

21. A method of manufacturing the medicament apparatus of claim 1, wherein the method is an additive manufacturing process and comprises a step of metal 3D printing and the additive manufacturing process is binder jetting of a metal powder.

22. (canceled)

23. The method of claim 21, wherein the metal powder's granule size is 50 microns or less.

24. The method of claim 21, wherein the metal powder's purity is at least 99.95%.

25. The method of claim 21, wherein sintering cycles that complete the binder jetting process are of low intensity such that voids in the internal porous matrix are mostly retained.

26. The method of claim 24, wherein prior to binder jetting a space holder material is mixed with the metal powder, the space holder material being subsequently removed by sintering thereby creating pores in the internal porous matrix.

27. (canceled)

28. The method of claim 26, wherein the space holder material is needlelike.

29. A method of treating or preventing infection in livestock animals using the medicament apparatus of claim 1, wherein the method comprises applying the medicament apparatus to a treatment site of the animal; and
(a) injecting the medicament apparatus into skin near a hoof of the animal to prevent or treat foot rot, digital dermatitis, inter-digital dermatitis, white line disease, and/or sole ulcers,
(b) inserting the medicament apparatus into a teat canal of the animal to prevent or treat mastitis and/or flystrike, to prevent contaminants from entering the teat canal, or as a cannula, teat straightener, internal teat canal skin conditioner, or teat end closure, or
(c) externally adhering the medicament apparatus to a wound of the animal to prevent or treat infection.

30-32. (canceled)

33. The method of claim 29, wherein the animal is a lactating animal selected from the group consisting of cow, goat, pig, deer, buffalo, sheep, and camel.

34. A medicament apparatus for use with animals, the medicament apparatus comprising:
a flexible and resilient mounting member made from a shape memory alloy;
one or more bodies each made from one or more solid metals, the metals selected from among those for which their ions have biocidal effect;
wherein the one or more bodies are mounted on the mounting member and an external surface of each of the bodies is configured to be situated at a treatment site of an animal;
wherein the medicament apparatus is formed as an elongate plug suitable for insertion into a teat canal of a lactating animal; and
wherein the mounting member is a shaft, and the one or more bodies are mounted along the length of the shaft.

35-36. (canceled)

37. The medicament apparatus of claim 34, wherein the shaft defines a widened region towards one end and the widened region is defined by adjacent branches of the shaft which diverge and reconverge configured to retain apparatus in the animal while still allowing drainage.

38. (canceled)

39. The medicament apparatus of claim 34, wherein the medicament apparatus further comprises an end stop fixed at an opposite end of the shaft.

40. The medicament apparatus of claim 34, wherein the one or more bodies comprise beads mounted on the shaft configured to facilitate flexing of the apparatus.

41. The medicament apparatus of claim 34, wherein the one or more bodies comprise wire wound about the shaft.

42-48. (canceled)
